(19) United States
(12) Patent Application Publication
(10) Pub. No.: US 2026/0183341 A1
(43) Pub. Date: Jul. 2, 2026

(54) USE OF COMPOSITIONS COMPRISING SMALL EXTRACELLULAR VESICLES DERIVED FROM UMBILICAL CORD BLOOD MONONUCLEAR CELLS FOR APPLICATION TO FIBROTIC LESIONS

(71) Applicant: EXOGENUS THERAPEUTICS, SA, Cantanhede (PT)

(72) Inventors: Joana CORREIA, Tentugal (PT); Patricia FREIRE, Ilhavo (PT); Tania LOURENCO, Cantanhede (PT)

(73) Assignee: EXOGENUS THERAPEUTICS, SA, Cantanhede (PT)

(21) Appl. No.: 19/130,555

(22) PCT Filed: Nov. 17, 2023
(86) PCT No.: PCT/IB2023/061635

§ 371 Date: May 16, 2025

(30) Foreign Application Priority Data
Nov. 18, 2022 (PT) PT118350

(51) Int. Cl. A61K 35/51; A61P 11/00; A61P 17/00

(52) U.S. Cl. CPC A61K 35/51; A61P 11/00; A61P 17/00

(57) ABSTRACT

The present invention relates to the use of compositions comprising specific exosomes secreted by umbilical cord blood mononuclear cells (UCBMNCs) in the prevention and treatment of fibrotic disorders such as fibrotic skin and lung injuries. The exosome compositions of the invention present activity in skin fibrosis and in lung diseases. Moreover, these compositions are also effective in reverting or preventing disease-associated parameters, such of lung diseases, proving its action against respiratory disorders with an inflammatory component, particularly when mediated by macrophages and/or T-cells. Therefore, the present invention lays in the technical domain of pharmaceuticals, medicine, cosmetics, dermo-cosmetics, research and development in cellular biology and appliances thereof.

### TECHNICAL FIELD

The present invention relates to the use of known compositions comprising specific exosomes secreted by umbilical cord blood mononuclear cells (UCBMNCs) in the prevention and treatment of fibrotic disorders such as fibrotic skin and lung injuries.

The exosome composition herein described as ExoCell of the present invention shows activity in for skin fibrosis and for lung disease.

Data support the use of these compositions for preventing and treating skin fibrotic disorders, including but not limited to scleroderma, and the known mechanism of action (MoA) and biodistribution pattern point to a potential beneficial effect in liver diseases, such as fibrosis. Moreover, said compositions are effective in reverting or preventing disease-associated parameters in in vivo models of lung disease, proving its potential for respiratory disorders with an inflammatory component, particularly when mediated by macrophages and/or T-cells. These may include the development of fibrosis following inflammatory stimuli such as infections, the management of chronic obstructive pulmonary disease (COPD), and the treatment of sarcoidosis.

Therefore, the present invention lays in the technical domain of pharmaceuticals, medicine, cosmetics, dermocosmetics, research and development in cellular biology and appliances thereof.

### BACKGROUND OF THE INVENTION

Exosomes are liposome-like vesicles (30-200 nm) secreted by most types of cells, which are formed inside the secreting cell in compartments referred as multivesicular bodies (MVBs) and are subsequently released by fusion of the endosomal compartment with the plasma membrane, resulting in content release to the extracellular milieu. They contain specific sets of proteins, lipids and RNA, depending on the type of secreting cell and stimuli, and not a random sample of cytoplasmic content. They carry genetic material, in the form of mRNA and microRNA, a feature that promoted them to promising biologically derived gene-delivery systems that can be used in therapeutic approaches.

Use of exosomes secreted by umbilical cord blood mononuclear cells (UCBMNCs) has been a widely discussed theme in literature and there are several documents already available.

Fibrosis is characterized by an overgrowth and scar formation in several tissues and is attributed to an excessive deposition of extracellular matrix (ECM) components.

Chronic inflammatory reactions induced by infections, chemical insults or tissue damage can cause fibrosis. Fibrosis can lead to organ failure and death. Some examples of fibrotic disorders are pulmonary fibrosis, liver cirrhosis, cardiovascular fibrosis, systemic sclerosis and nephritis.

Pulmonary fibrosis is described as a chronic lung disease characterized by an abnormal accumulation of extracellular matrix (ECM) and remodelling of lung architecture.

Lung fibrosis caused by scar formation can destroy the lung architecture in a progressive and irreversible manner that ultimately can lead to lung malfunction, disruption of gas exchange, and death from respiratory failure. Pulmonary fibrosis is refractory to treatment and has high mortality rates. Idiopathic pulmonary fibrosis (IPF) is a chronic fibrotic lung disease of unknown cause and occurs in middle-aged adults. IPF is related with a poor prognosis.

IPF is one of the most common lung diseases encountered in pulmonary practices. The incidence of IPF ranges between 0.22 and 7.4 per 100,000 population in Europe and ranges between 16.3-17.4 per 100,000 population in United States of America. Prevalence and incidence of IPF increase with age and appear to be on the increase in recent years.

Pulmonary fibrosis mouse model involves the delivery of a single dose of bleomycin (BLM) intratracheal or intranasal to lung. Bleomycin induces lung damage characterized by inflammatory cell infiltrates, collagen deposition and fibrosis.

Acute lung injury mouse model involves the administration of lipopolysaccharide (LPS) to the airways that causes an acute inflammatory reaction, with massive cellular infiltration to the lungs.

Document WO2017163132 describes compositions comprising UCBMNCs exosomes for tissue repair, in particular for wound treatment and respective process for obtaining these exosomes and compositions thereof.

Document WO2020070700 describes enriched umbilical cord blood mononuclear cells (UCBMNCs) Small Extracellular Vesicles (SEVs) and an optimized process of obtaining them with application to autoimmune diseases associated with inflammation.

However, none of the cited prior art uses UCBMNCs exosomes to be applied to fibrotic disorders such as fibrotic lung injury. Therefore, there is a need to develop innovative compositions and methods to be applied as therapy for fibrosis.

For this purpose, the present invention provides exosome compositions (ExoCell) as disclosed in documents WO2017163132 and WO2020070700 comprising specific UCBMNCs derived exosomes for treatment or prevention of fibrotic diseases or disorders, namely skin and lung disorders.

### BRIEF DESCRIPTION OF FIGURES

FIG. 1 Represents the expression of ACTA2 mRNA in normal human dermal fibroblasts (NHDF) cells non-treated (NT), treated with TGF-β1, with the exosome compositions of the invention (ExoCell) or PBS by RT-PCR analysis of ACTA2. Data represent mean±SD. Statistical analysis using unpaired t-test.

FIG. 2A Representative microscope images of normal human dermal fibroblasts (NHDF) immuno-stained against α-SMA in non-treated (-TGFβ1), treated with TGF-β1 (+TGFβ1).

FIG. 2B Presents the mean fluorescence intensity (MFI) of α-SMA in non-treated and treated NHDF cells. Data represent mean±SD. Statistical analysis using unpaired t-test.

FIG. 3A Representative microscope images of normal human dermal fibroblasts (NHDF) immune-stained against α-SMA in non-treated (-TGFβ1), treated with TGF-β1 and vehicle (+TGFβ1 vehicle-PBS) and treated with TGF-β1 and ExoCell.

FIG. 3B Presents the fluorescence intensity (FI) measured along of the transects transversal to the cells (represented in FIG. 3a) non-treated, treated with TGFβ and vehicle (PBS) and treated with TGFβ and ExoCell. Data represents the mean of the FI from the transects of 12 cells (represented in FIG. 3a).

FIG. 3C Presents the fold change of MFI to the vehicle and non-treated and ExoCell treated. Data represent mean±SD. Statistical analysis using one-sample t-test. *p≤0.05, **p≤0.01

FIG. 4 Presents the distribution of ExoCell by total radiant efficiency ([p/s]/[μW/cm²]) in lungs and stomach 1 hour after intranasal (IN), intratracheal (IT) or microsprayer-coupled IT administrations of labelled PBS (Control) or fluorescently labelled ExoCell of the invention in C57BL6 male mice. The organ images were taken together by IVIS-Spectrum. Results are expressed as mean±SD (n=3 for Control and n=5 for the exosome compositions treated groups). *p≤0.05. Statistical analysis 2-way ANOVA, Tukey's multiple comparison post-test.

FIG. 5 Presents the effect of the composition of the invention ExoCell in the recovery of weight in the tested mice in assays having a dose-dependent fashion.

FIG. 5A Shows the evolution of body weight along the experiment in healthy mice (blank), mice treated with ExoCell at doses A or B, wherein PBS is used as negative control and nintedanib (clinical comparator) Data are expressed as Mean±SD of 9-12 mice per group. Statistical evaluation of differences between the experimental groups was determined by using two-way ANOVA followed by a Sidak's multiple comparisons post-test or Tukey's multiple comparisons post-test. p values≤0.05 were considered significant.

FIG. 5B Shows the collagen concentration in lung homogenates at day 14. Collagen concentration (μg/mL) increased in mice treated with Bleomycin (BLM).

FIG. 5C Ashcroft score was evaluated at D14. Ashcroft score decreased upon ExoCell treatment in the dose B group (1×10¹⁰ particles). Data are expressed as Mean±SD of 8-12 mice per group. Statistical evaluation of differences between the experimental groups was determined by using a nonparametric Kruskal-Wallis followed by Dunn's Multiple comparison test. p values≤0.05 were considered significant. ***p≤0.001; ****p≤0.0001.

FIG. 6 Shows the effect of the ExoCell in the weight loss and collagen accumulation.

FIG. 6A Shows the evolution of body weight along the experiment in healthy mice (blank) or mice treated with the compositions of the invention, dose B, wherein PBS is the negative control, or nintedanib as clinical comparator. Data are expressed as Mean±SD of 9-12 mice per group. Statistical evaluation of differences between the experimental groups was determined by using two-way ANOVA followed by a Sidak's multiple comparisons post-test and Tukey's multiple comparisons post-test. p values≤0.05 were considered significant.

FIG. 6B Presents the collagen concentration in lung homogenates at D14. Collagen concentration (μg/mL) increased in mice treated with BLM and this accumulation was reduced or prevented by administration ExoCell of the invention (dose B, 1×10¹⁰ particles).

FIG. 6C Ashcroft score was evaluated at D14. Ashcroft score decreased upon ExoCell treatment in dose B (1×10¹⁰ particles). Data are expressed as Mean±SD of 8-12 mice per group. Statistical evaluation of differences between the experimental groups was determined by using a nonparametric Kruskal-Wallis followed by Dunn's Multiple comparison test. p values≤0.05 were considered significant.

FIG. 7 Shows that the IV administration of the compositions of the invention targets T-cells.

FIG. 7A Total number of T-cells in BALF.

FIG. 7B Number of Teff (CD8+) cells in BALF.

FIG. 7C Number of T-helper (CD4+) cells in BALF.

FIG. 7D Percentage of total T-cells in BALF

FIG. 7E Percentage of Teff (CD8+) cells in BALF

FIG. 7F Percentage of T-helper (CD4+) cells in BALF. T-cells were identified as CD45+CD3+ single live lymphocytes, and further separated into CD4+ or CD8+ cells.

Data are expressed as Mean±SD of 8-12 mice per group. Statistical evaluation of differences between the experimental groups was determined by using a non-parametric Kruskal-Wallis followed by Dunn's Multiple comparison test. p values≤0.05 were considered significant. *p≤0.05, **p≤0.01, ***p≤0.001, p≤0.0001.

FIG. 8 Presents the absolute cell counts for each leukocyte subset and IL-10 concentration in BALF from acute lung injury mouse model.

FIG. 8A Total cells in BALF, and

FIG. 8B Total cell counts of neutrophils,

FIG. 8C Total cell counts of lymphocytes, and

FIG. 8D Total cell counts of macrophages in BALF.

FIG. 8E IL-10 concentration (pg/mL) in BALF.

Data represent mean±SD (n=9). *p≤0.05, **p≤0.01, ***p≤0.001 and ****p≤0.0001.

Statistical differences were determined by using one-way ANOVA and Bonferroni post-test.

FIG. 9—Shows the Cytokine levels in BALF from acute lung injury mouse model,

FIG. 9A IL-6 concentration (pg/mL),

FIG. 9B TNF-α concentration (pg/mL),

FIG. 9C IL17A concentration (pg/mL), and

FIG. 9D IFNγ (pg/mL).

Data represent mean±SD (n=9). *p≤0.05, **p≤0.01, ***p≤0.001 and ****p≤0.0001.

Statistical differences were determined by using one-way ANOVA and Tukey post-test.

### DESCRIPTION OF THE INVENTION

The present invention relates to the use of compositions comprising specific exosomes secreted by umbilical cord blood mononuclear cells (UCBMNCs) in the prevention and treatment of fibrotic disorders such as fibrotic skin and lung injuries.

The specific exosomes secreted by UCBMNCs and compositions thereof hereinafter referred as ExoCell, are prepared according to the method described in the document WO2017163132 or by the optimized method as described in the document WO2020070700.

In a preferred embodiment the exosomes and compositions thereof of the present invention are prepared according to the optimized method as described in the document WO2020070700.

The exosomes of the present invention can be lyophilized as described in the document WO2020070700 in order to produce a storable product and/or to produce compositions.

The exosomes of the present invention (ExoCell) contain specific sets of proteins, lipids and RNA, that carry genetic material, in the form of mRNA and microRNA, a feature that promoted them to promising biologically derived gene-delivery systems that can be used in therapeutic approaches.

The exosomes compositions used in the present invention comprise one or more bioactive molecules selected from the following (a) proteins, (b) miRNAs and (c) lipids as in the lists below:

### Proteins:

| | UniProt | |
|---|---|---|
| Nr. | ID | Name |
| 1 | P0DOX2 | IGA2 |
| 2 | P0DOX7 | IGK |
| 3 | P01871 | IGHM |
| 4 | P01619 | KV320 |
| 5 | Q5VTE0 | EF1A3 |
| 6 | P01876 | IGHA1 |
| 7 | P04217 | A1BG |
| 8 | P01023 | A2M |
| 9 | P63261 | ACTG1 |
| 10 | P12814 | ACTN1 |
| 11 | P43652 | AFM |
| 12 | P02765 | AHSG |
| 13 | P02768 | ALB |
| 14 | P04075 | ALDOA |
| 15 | P02760 | AMBP |
| 16 | P04083 | ANXA1 |
| 17 | P50995 | ANXA11 |
| 18 | P07355 | ANXA2 |
| 19 | P12429 | ANXA3 |
| 20 | P08758 | ANXA5 |
| 21 | P08133 | ANXA6 |
| 22 | P20073 | ANXA7 |
| 23 | O75882 | ATRN |
| 24 | P25311 | AZGP1 |
| 25 | P30043 | BLVRB |
| 26 | P02745 | CIQA |
| 27 | P02746 | C1QB |
| 28 | P02747 | C1QC |
| 29 | P09871 | C1S |
| 30 | P01024 | C3 |
| 31 | P04003 | C4BPA |
| 32 | P02748 | C9 |
| 33 | P00918 | CA2 |
| 34 | P04040 | CAT |
| 35 | Q08722 | CD47 |
| 36 | P08962 | CD63 |
| 37 | P23528 | CFL1 |
| 38 | P10909 | CLU |
| 39 | P31146 | CORO1A |
| 40 | P00450 | CP |
| 41 | P01040 | CSTA |
| 42 | P08311 | CTSG |
| 43 | P81605 | DCD |
| 44 | P59666 | DEFA3 |
| 45 | Q02413 | DSG1 |
| 46 | P15924 | DSP |
| 47 | P60842 | EIF4A1 |
| 48 | P08246 | ELANE |
| 49 | P06733 | ENO1 |
| 50 | P16452 | EPB42 |
| 51 | P21333 | FLNA |
| 52 | P02751 | FN1 |
| 53 | P04406 | GAPDH |
| 54 | P02774 | GC |
| 55 | P13224 | GP1BB |
| 56 | P06396 | GSN |
| 57 | O15511 | ARPC5 |
| 58 | P46976 | GYG1 |
| 59 | P69905 | HBA1 |
| 60 | P68871 | HBB |
| 61 | P02042 | HBD |
| 62 | P69891 | HBG1 |
| 63 | P69892 | HBG2 |
| 64 | P02008 | HBZ |
| 65 | P62805 | HIST4H4 |
| 66 | P00738 | HP |
| 67 | P02790 | HPX |
| 68 | Q86YZ3 | HRNR |
| 69 | P11142 | HSPA8 |
| 70 | P08514 | ITGA2B |
| 71 | P05106 | ITGB3 |
| 72 | P01591 | JCHAIN |
| 73 | P14923 | JUP |
| 74 | P01042 | KNG1 |
| 75 | P11279 | LAMP1 |
| 76 | P31025 | LCN1 |
| 77 | P07195 | LDHB |
| 78 | Q08380 | LGALS3BP |
| 79 | P02750 | LRG1 |
| 80 | P02788 | LTF |
| 81 | P61626 | LYZ |
| 82 | Q08431 | MFGE8 |
| 83 | P05164 | MPO |
| 84 | P35579 | MYH9 |
| 85 | P60660 | MYL6 |
| 86 | Q6UX06 | OLFM4 |
| 87 | P07737 | PFN1 |
| 88 | P14618 | PKM |
| 89 | P55058 | PLTP |
| 90 | P02775 | PPBP |
| 91 | P62937 | PPIA |
| 92 | P32119 | PRDX2 |
| 93 | P30041 | PRDX6 |
| 94 | P05109 | S100A8 |
| 95 | P06702 | S100A9 |
| 96 | P29508 | SERPINB3 |
| 97 | P05155 | SERPING1 |
| 98 | P11166 | SLC2A1 |
| 99 | P02730 | SLC4A1 |
| 100 | P11277 | SPTB |
| 101 | P30626 | SRI |
| 102 | P27105 | STOM |
| 103 | P02787 | TF |
| 104 | P02786 | TFRC |
| 105 | P07996 | THBS1 |
| 106 | Q9Y490 | TLN1 |
| 107 | P24821 | TNC |
| 108 | P60174 | TPI1 |
| 109 | P62987 | UBA52 |
| 110 | P13611 | VCAN |
| 111 | P18206 | VCL |
| 112 | P31946 | YWHAB |
| 113 | P63104 | YWHAZ |
| 114 | P0DOX5 | IGG1 |
| 115 | P16157 | ANK1 |
| 116 | P0COL5 | C4B |
| 117 | P31944 | CASP14 |
| 118 | Q16778 | HIST2H2BE |
| 119 | P01859 | IGHG2 |
| 120 | Q05707 | COL14A1 |
| 121 | Q01518 | CAP1 |
| 122 | P02549 | SPTA1 |
| 123 | P16403 | HIST1H1C |
| 124 | P00736 | C1R |
| 125 | P0DOY2 | IGLC2 |
| 126 | P12273 | PIP |
| 127 | Q96P63 | SERPINB 12 |
| 128 | P07478 | PRSS2 |
| 129 | P02649 | APOE |
| 130 | Q14254 | FLOT2 |
| 131 | Q08188 | TGM3 |
| 132 | P67936 | TPM4 |
| 133 | Q06830 | PRDX1 |
| 134 | 043866 | CD5L |
| 135 | P08238 | HSP90AB1 |
| 136 | Q9NZD4 | AHSP |
| 137 | P01768 | HV330 |
| 138 | Q71DI3 | HIST2H3C |
| 139 | P40926 | MDH2 |
| 140 | P19338 | NCL |
| 141 | Q93077 | HIST1H2AC |
| 142 | P01031 | C5 |
| 143 | P20618 | PSMB1 |
| 144 | O00299 | CLIC1 |
| 145 | P09105 | HBQ1 |
| 146 | Q9UL46 | PSME2 |
| 147 | Q86TJ2 | TADA2B |
| 148 | Q16610 | ECM1 |
| 149 | P60900 | PSMA6 |
| 150 | P53396 | ACLY |
| 151 | P37802 | TAGLN2 |
| 152 | P35754 | GLRX |
| 153 | P29972 | AQP1 |
| 154 | P28072 | PSMB6 |
| 155 | P28066 | PSMA5 |
| 156 | P06331 | HV434 |
| 157 | O75635 | SERPINB7 |
| 158 | O75368 | SH3BGRL |
| 159 | P59190 | RAB15 |
| 160 | Q08554 | DSC1 |
| 161 | P01743 | HV146 |
| 162 | P80188 | LCN2 |
| 163 | P05089 | ARG1 |
| 164 | O14818 | PSMA7 |
| 165 | Q6ZVX7 | NCCRP1 |
| 166 | P19652 | ORM2 |
| 167 | P48059 | LIMS1 |
| 168 | P29353 | SHC1 |
| 169 | P10599 | TXN |
| 170 | Q5T749 | KPRP |

more preferably one or more proteins selected from the group of ANXA2, ANK1, CD63, CD81, CD9, CD15, more preferably wherein said proteins are present in the SEVs compositions in the amounts of: CD81≥1%, CD9≥1%, CD63≥40%, CD15≥20% positive events as measured by flow cytometry of SEVs coupled to microbeads, or quantity of CD63≥5 μg/mL or ANXA2≥0.3 ng/mL in the purified SEVs, as measured by ELISA;

b) miRNAs:

| | | |
|---|---|---|
| hsa-let-7a-5p | hsa-miR-15a-5p | hsa-miR-21-5p |
| hsa-let-7f-5p | hsa-miR-15b-5p | hsa-miR-223-3p |
| hsa-let-7g-5p | hsa-miR-16-5p | hsa-miR-23a-3p |
| hsa-miR-103a-3p | hsa-miR-17-5p | hsa-miR-26a-5p |
| hsa-miR-106b-5p | hsa-miR-191-5p | hsa-miR-26b-5p |
| hsa-miR-142-3p | hsa-miR-19a-3p | hsa-miR-29b-3p |
| hsa-miR-146a-5p | hsa-miR-19b-3p | hsa-miR-30d-5p |
| hsa-miR-150-5p | hsa-miR-20a-5p | hsa-miR-451a |
| hsa-let-7b-5p | hsa-miR-185-5p | hsa-miR-30b-5p |
| hsa-let-7c-5p | hsa-miR-18a-5p | hsa-miR-3184-3p |
| hsa-let-7i-5p | hsa-miR-205-5p | hsa-miR-376c-3p |
| hsa-miR-130a-3p | hsa-miR-221-3p | hsa-miR-486-5p |
| hsa-miR-144-3p | hsa-miR-22-3p | hsa-miR-92a-3p |
| hsa-miR-144-5p | hsa-miR-27a-3p | hsa-miR-93-5p |
| hsa-miR-181a-5p | hsa-miR-27b-3p | |

preferably comprising one or more of the following miRNAs: hsa-let-7b-5p, hsa-let-7c-5p, hsa-let-7i-5p, hsa-miR-130a-3p, hsa-miR-144-13p, hsa-miR-144-5p, hsa-miR-181a-5p, hsa-miR-185-5p, hsa-miR-18a-5p, hsa-miR-205-5p, hsa-miR-221-13p, hsa-miR-22-3p, hsa-miR-27a-3p, hsa-miR-27b-3p, hsa-miR-30b-5p, hsa-miR-3184-13p, hsa-miR-376c-3p, hsa-miR-486-5p, hsa-miR-92a-3p and hsa-miR-93-5p, more preferably miR-150-5p, miR-223-3p, miR-16-5p, miR-142-3p, miR-19b,

and more preferably wherein said miRNAs are present in the SEVs composition in the respective amounts of: miR-150-5p>1.3 pg/10\hat{ }9part, miR-223-3p>1.2 pg/10\hat{ }9part, miR-16-5p>0.5 pg/10\hat{ }9part, miR-142-3p>0.4 pg/10\hat{ }9part, miR-19b>0.2 pg/10\hat{ }9part, as measured by direct quantification, and

c) lipids: cholesteryl esters (CE), Diacylglycerides (DAG), phosphatidic acid (PA), phosphatidylcholines (PC), phosphatidylethanolamine (PE), phosphatidylglycerol (PG), phosphatidylinositol (PI), phosphatidylserines (PS), Sphingomyelin (SPM), Triacyl glycerides (TAG), preferably phosphatidylcholines (PC), phosphatidylserines (PS) and Sphingomyelin (SPM), and more preferably wherein said lipids are present in the SEVs composition in a minimum percentage, respective to the total lipid amount of: CE≥0.05±0.05, DAG≥1.3±0.4, PA≥3.18±0.6, PC≥30.9±3.3, PE≥13.0±0.5, PG≥0.25±0.1, PI≥3.8±0.3, PS≥29.4±3.7, SPM≥18.0±1.6, TAG≥0.074±0.13.

In one embodiment, the exosome compositions ExoCell comprise:

proteins that consist in combinations of the presented proteins, as listed above, preferably in combinations of proteins selected from the group of ANXA2, ANK1, CD63, CD81, CD9, CD15, more preferably wherein the following proteins are present in the SEVs in the amounts of: CD81≥1%, CD9≥1%, CD63≥40%, CD15≥20% positive events as measured by flow cytometry of SEVs coupled to microbeads, or quantity of CD63≥5 μg/mL or ANXA2≥0.3 ηg/mL in the purified SEVs, as measured by ELISA;

miRNA that consist in combinations of the presented miRNA, as listed above, preferably in combinations of miRNAs selected from the following: miR-150-5p≥1.3 pg/10\hat{ }9part, miR-223-3p≥1.2 pg/10\hat{ }9part, miR-16-5p≥0.5 pg/10\hat{ }9part, miR-142-3p≥0.4 pg/10\hat{ }9part, miR-19b≥0.2 pg/10\hat{ }9part, as measured by direct quantification, and

c) lipids that consist in combinations of the presented lipids, as listed above, preferably in combinations of lipids selected from CE, Diacylglycerides (DAG), PA, phosphatidylcholines (PC), PE, PG, PI, phosphatidylserines (PS), Sphingomyelin (SPM), Triacylglycerides (TAG), more preferably phosphatidylcholines (PC), phosphatidylserines (PS) and Sphingomyelin (SPM), and more preferably in the following respective percentages, respective to the total lipid amount of: CE≥0.05±0.05, DAG≥1.3±0.4, PA≥3.18±0.6, PC≥30.9±3.3, PE≥13.0±0.5, PG≥0.25±0.1, PI≥3.8±0.3, PS≥29.4±3.7, SPM≥18.0±1.6, TAG≥0.074±0.13.

In another embodiment, the exosome composition ExoCell comprises the following combination of proteins, miRNAs and lipids:
Proteins: ANXA2, CD63, CD81, CD9 and CD15;
RNAs: preferably miR-150-5p, miR-223-3p, miR-16-5p, miR-142-3p, miR-19b, and
Lipids: CE≥0.05±0.05, DAG≥1.3±0.4, PA≥3.18±0.6, PC≥30.9±3.3, PE≥13.0±0.5, PG≥0.25±0.1, PI≥3.8±0.3, PS≥29.4±3.7, SPM≥18.0±1.6, TAG≥0.074±0.13.

In another embodiment, the exosome composition ExoCell comprises exosomes that are enriched with miRNAs, palmitoylated tripeptides, peptides, DNA, siRNA, growth factors, amino acids, sugars, lipid soluble molecules, fatty acids and its derivatives such as DHA, EPA, Oleic acid, lipid modified molecules such as GPI anchored proteins or peptides, and/or other hydrophobic molecules, preferably SEVs are enriched miRNAs, or palmitoylated tripeptides.

In another embodiment, the exosome composition ExoCell comprises exosomes that are enriched with one or more miRNAs selected from a group consisting of: hsa-miR-150-5p, hsa-miR-16-5p, hsa-miR-142-3p, hsa-miR-223-3p, hsa-let-7g-5p, hsa-miR-21-5p, hsa-let-7f-5p, hsa-miR-19b-3p, hsa-let-7a-5p, hsa-miR-26a-1-5p, hsa-miR-20a-5p, hsa-miR-181a-5p, hsa-miR-451a, hsa-miR-23a-3p, hsa-miR-342-3p, hsa-miR-191-5p, hsa-miR-103a-3p, hsa-miR-15a-5p, hsa-miR-142-5p, hsa-miR-146a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p,

preferably the exosomes are enriched with one or more miRNAs selected from a group consisting of: hsa-miR-181a-5p, hsa-miR-451a, hsa-miR-103a-3p, hsa-miR-15a-5p, hsa-miR-19a-3p, hsa-miR-15b-5p, hsa-miR-26b-5p, hsa-miR-30d-5p, hsa-miR-146b-5p, hsa-miR-106b-5p, hsa-miR-29a-3p, hsa-miR-17-5p, hsa-miR-29b-3p, and hsa-miR-101-3p,

preferably the exosomes are enriched with miRNAs hsa-miR-150-5p, thereby increasing the concentration of miRNAs in the exosome composition.

In vitro model developed to treat skin fibrosis (dermal fibroblasts treated with TGF-β1—transforming growth factor-beta 1) showed a decreased expression of α-SMA (alpha smooth muscle actin)—a marker of fibrosis upon—treatment with the exosome compositions of the invention. These results indicate a positive effect of said compositions in fibrosis reduction.

In the context of lung disorders, in vivo models developed to evaluate the exosome compositions activity and mechanism of action (MoA), namely lipopolysaccharide (LPS)-induced acute lung inflammation (ALI) and bleomycin (BLM)-induced lung fibrosis, both in rodents.

Exosome compositions (dose dependently) administered locally (intratracheally, IT) or systemically (intravenous, IV) to IPF model accelerated their overall recovery or prevented overt disease, as measured by weight changes, and reduced lung fibrosis (evaluated biochemically by collagen accumulation in the lung and histologically using the Ashcroft score).

In vivo, local administration of exosome compositions (intranasally, IN) was shown to specifically target macrophages in a model of LPS-induced acute lung inflammation, leading to the normalization of their numbers in bronchoalveolar lavage fluid (BALF). Additionally, exosome compositions reduced the concentration of pro-inflammatory mediators in BALF, such as IL-1b and IL-17A, in a dose-response manner.

Available data point to a macrophage- and/or T-cell-dependent MoA, determined by the disease context and compound's administration route.

Finally, considering the natural biodistribution of exosome compositions to the liver and the influence of macrophages on hepatic fibrotic processes, said compositions are potentially effective in the prevention, deceleration, or reversion of liver fibrosis.

The exosome compositions of the present invention (ExoCell) showed activity in one in vitro model for skin fibrosis and two in vivo models of lung disease. In vitro data support the use of said compositions for skin fibrotic disorders, including but not limited to scleroderma, and the known MoA and biodistribution pattern point to a potential beneficial effect in liver diseases, such as fibrosis. Moreover, these compositions are effective in reverting or preventing disease-associated parameters in two in vivo models of lung disease, proving its potential for respiratory disorders with an inflammatory component, particularly when mediated by macrophages and/or T-cells. These may include the development of fibrosis following inflammatory stimuli such as infections, the management of COPD, and the treatment of sarcoidosis.

Accordingly, the present invention also relates to pharmaceutical compositions comprising the ExoCell compositions as described above in a liquid form, in a suspension form, in a powder form, in a spray form, in a cream form, in a gel form or hydrogel, form of aerosol, vapour for inhalation and nebulization.

In another embodiment, the above-mentioned pharmaceutical composition is a medical device supported by a liquid form, in a suspension form, in a powder form, in a spray form, in a cream form, in a gel form or hydrogel, form of aerosol, vapour for inhalation and nebulization.

The exosome compositions and pharmaceutical compositions thereof are useful in the prevention and treatment of fibrotic disorders.

In one embodiment, the fibrotic disorders are fibrotic lesions associated with inflammation, autoimmune diseases, infections, injury, or idiopathic disorders.

In this context, inflammation includes inflammatory diseases with skin fibrosis manifestations such as keloids and scars.

The autoimmune diseases include skin fibrosis manifestations such as scleroderma.

The lung viral or bacterial infections are associated with lung fibrosis such as acute respiratory distress syndrome (ARDS).

The injury disorders include skin fibrosis manifestations such as keloids and scars.

Fibrotic lesions may include lesions with or without autoimmune and/or inflammatory cause, such as systemic sclerosis, nephrogenic systemic fibrosis and, liver fibrosis.

The idiopathic diseases include lung fibrosis such as idiopathic pulmonary fibrosis (IPF).

Contrarily to the described in document WO2017163132 and document WO2020070700, which are also related with UCBMNCs exosomes and their use in therapeutic or prevention applications respectively for wound treatment by topic administration and for application to autoimmune diseases associated with inflammation by topic application or by subcutaneous, dermal, intraperitoneal and intravenous administration, the exosomes and compositions of this invention (ExoCell) are applied differently to produce the advantageous effects in the treatment or prevention of the fibrotic disorders as mentioned above, namely the fibrotic lesions associated with inflammation, autoimmune diseases, infections, injury, or idiopathic disorders by intravenous application or locally, e.g. by intratracheal, intranasal or local injection using for this purpose the ExoCell compositions in the form of aerosol, vapour inhalation, nebulization and liquid formulations.

Accordingly, in one embodiment for treating and prevention of idiopathic pulmonary fibrosis (IPF) conditions, the administration is preferably made via intratracheal instillation by introducing the compositions directly in the trachea by inserting a needle in the mouth and throat, or intravenous.

In another embodiment, intranasal administration is adequate for treating and prevention of acute lung injury conditions.

### EXAMPLES

### Example 1. Functional Effects of the Exosome Composition NE

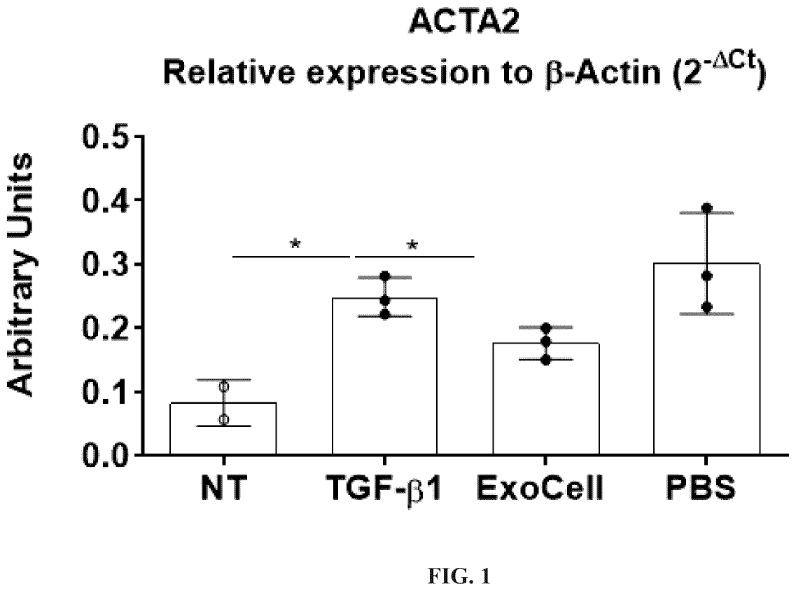

In vitro studies using a skin fibrotic model, primary normal human dermal fibroblasts (NHDF)—isolated from the dermis of juvenile foreskin or adult skin—treated with transforming growth factor beta 1 (TGF-β1) at 10 ng/ml to induce fibrosis (TGF-β1 is a central mediator of fibrogenesis)(Branton and Kopp 1999; Aoudjehane et al. 2016). NHDF cells were co-incubated with TGF-β1 and ExoCell (1×10¹⁰ particles/ml) for 3 times (day 0, day 3 and day 5). After 12 hours of the last treatment cells were collected to assess the expression of ACTA2 mRNA—alpha smooth muscle actin (α-SMA, a marker of fibrosis) by qPCR (3 independent experiments were performed). RNA samples were extracted by RNAeasy Mini kit (Qiagen) according to manufacture recommendation. RNA concentration was quantified using Nanodrop. All RNA samples were stored at −80° C. RNA was reverse transcribed according to manufacturer's instructions of SuperScript IV VILO Master Mix (Invitrogen). After reverse transcription, the cDNA was immediately used for qPCR or were preserved at −20° C. qPCR was performed using the NZYSpeedy qPCR Green Master Mix (2×), ROX in a 96-well format in CFX96 Touch™ Real-Time PCR Detection System (BioRad). The following conditions, 95° C. for 2 min, followed by 40 cycles at 95° C. for 5 s, 60° C. for 30 s, 72° C. for 20 s (measuring the fluorescence) were used for the qPCR. At least three biological replicates were used. p-actin (ACTB) was used as an endogenous control to normalize each sample. The resulting data was analysed using Bio-Rad CFX Manager software. The relative expression of interest genes was analysed according to the 2^{-ΔΔct} method. The expression of ACTA2 (normalized to P-actin) increased upon TGF-β1 treatment (P<0.05), as expected, and decreased when ExoCell was co-incubated with TGF-β1 (P<0.05), indicating a role of ExoCell in reducing fibrosis (FIG. 1) through decreasing ACTA2 expression.

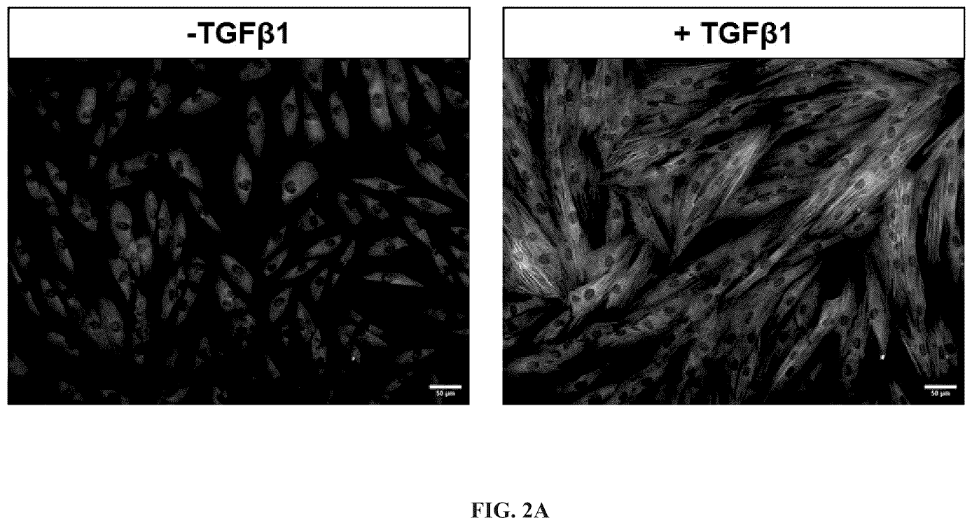

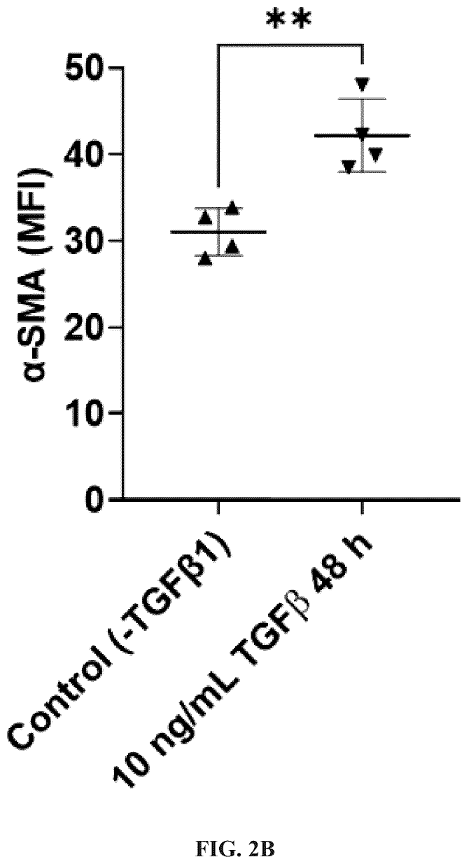

NHDF were harvested using trypsin and, plated at 20,000 cells/cm² in growth medium for 24 hours. After 24 hours, growth medium was replaced by serum free medium. In the following day, cells were treated with 10 ng/mL of TGFβ1 diluted in serum free medium. After 48 hours of TGFβ1 treatment, cells were crosslinked using 4% paraformaldehyde for 20 minutes, then blocked in 0.1% BSA in a solution of 0.1% PBS-triton for 30 minutes. Next, cells were immunostained against α-SMA (mouse) antibody diluted in blocking solution for 3 hours., cells were washed using 0.05% PBS-Tween for 3 times and, incubated in a blocking solution containing a fluorescent conjugated secondary antibody anti-mouse for 1 h. Then, cells were washed three times and nuclei were stained using DAPI (1 μg/mL). Images were acquired using a AxioVert microscope (FIG. 2 a).

Mean fluorescence intensity was measured using ImageJ. Briefly, MFI was measured by selecting cells with the Threshold function and measuring the fluorescence intensity. Then, the background intensity was subtracted to obtain the MFI (FIG. 2 b).

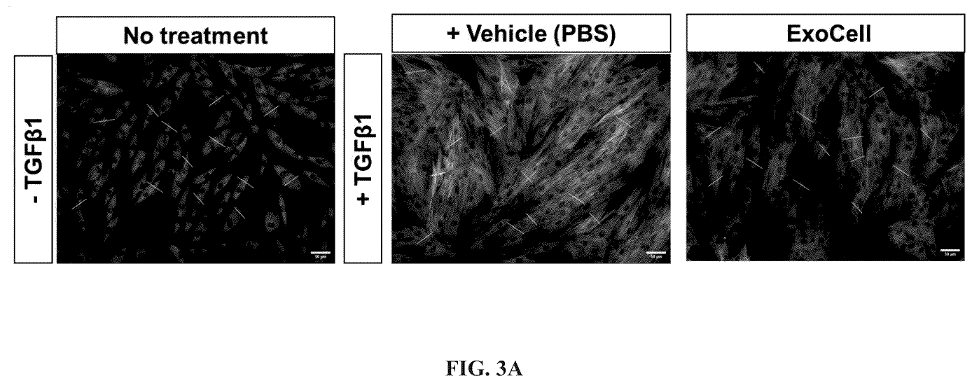

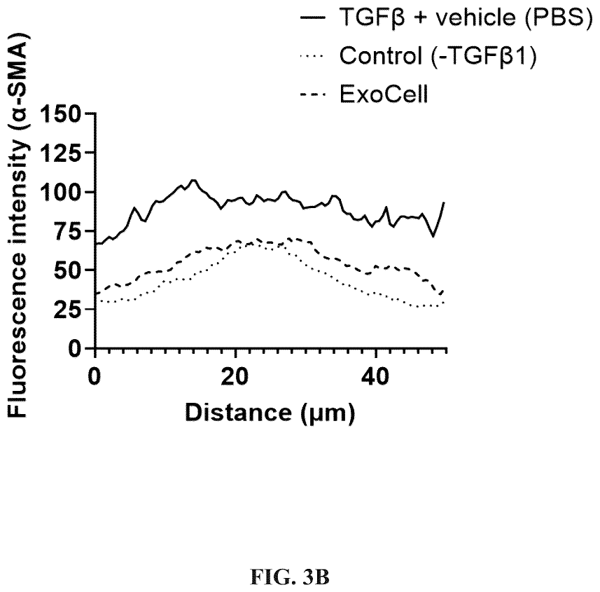

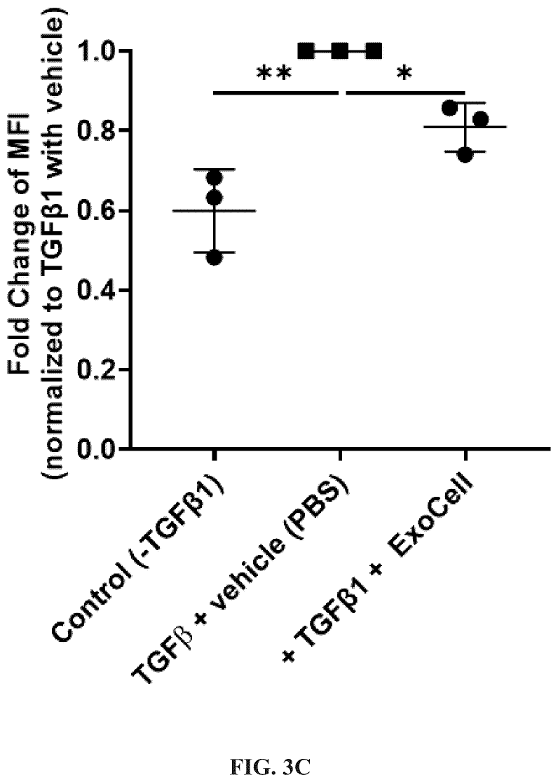

NHDF were harvested using trypsin and, plated at 20,000 cells/cm² in growth medium for 24 hours. After 24 hours, growth medium was replaced by serum free medium and, the pre-treated with ExoCell (20000 particles/cell). In the following day, cells were treated with 10 ng/mL of TGFβ1 diluted in serum free medium and with a second dose of ExoCell. After 48 hours of TGFβ1 treatment, cells were crosslinked using 4% paraformaldehyde for 20 minutes, then blocked in 0.1% BSA in a solution of 0.1% PBS-triton for 30 minutes. Next, cells were immunostained against α-SMA (mouse) antibody diluted in blocking solution for 3 hours, cells were washed using 0.05% PBS-Tween for 3 times and, incubated in a blocking solution containing a fluorescent conjugated secondary antibody anti-mouse for 1 h. Then, cells were washed three times and nuclei were stained using DAPI (1 μg/mL). Images were acquired using an AxioVert microscope. (FIG. 3a) Mean fluorescence intensity was measured using ImageJ. Briefly, MFI was measured by selecting cells with the Threshold function and measuring the fluorescence intensity. Then, the background intensity was subtracted to obtain the MFI. The fluorescence intensity (FI) of the fibers was measured by drawing transversal lines crossing all cell, then ImageJ software measured the FI along the cut. These were performed in 12 cells and, then the mean was plotted using GraphPad (FIG. 3b). MFI of control non-treated and ExoCell treated cells was normalized to MFI from cells treated with TGFβ 1 cells treated with PBS.

### Example 2. Functional Effects of ExoCell in Lung Fibrosis

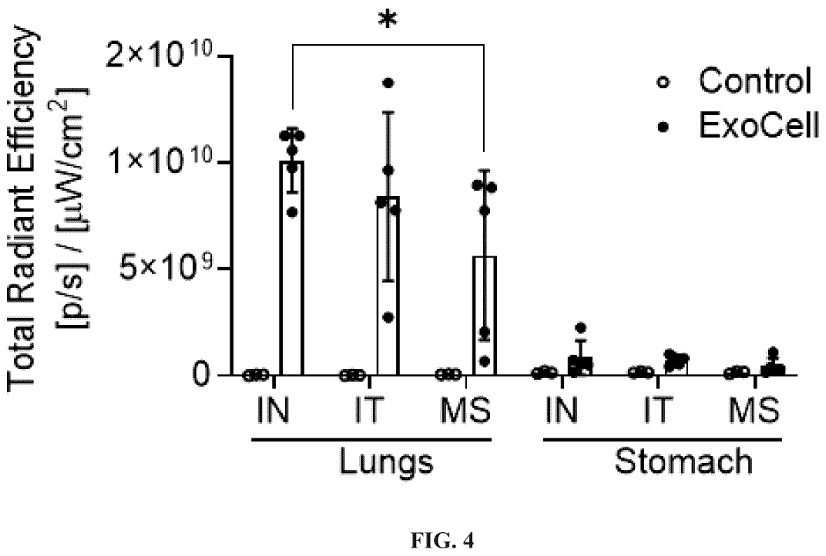

As a proof of concept of exosomes delivery to the lungs, exosome compositions were labelled with DiR (1,1-dioctadecyl-3,3,3,3-tetramethylindotricarbocyanine iodide; D12731, Thermo Fisher Scientific) fluorophore on the day before their administration to the mice and were stored at 4° C. and protected from the light until day 1. After labelling exosomes, an aliquot was used to determine fluorescence on IVIS (black bottom 96-well plates). Animals were treated with either vehicle (Blank, dye exposed PBS, 3 animals) or fluorescently labelled exosome composition administered (5 animals per condition) intranasally, intratracheally and with microsprayer aerosolizer. Fifty μL of ExoCell solution (1×10¹¹ particles/mL, total of 5×10⁹) were administered. Blank animals were intranasally, intratracheally and with microsprayer aerosolizer administered with 50 μL of vehicle (labelled PBS) respectively. One hour after the composition administration animals were sacrificed, and individual lungs and stomachs were collected to measure fluorescence by IVIS-Spectrum In Vivo Imaging System (Perkin Elmer). Fluorescence was measured in organs separately. Both IN and IT administration routes resulted in a homogenous distribution of ExoCell in the lung (FIG. 4). Microsprayer-coupled IT (MS) administration proved unsuitable, due to lower particle intensity and high variability.

### 2.1—Pulmonary Fibrosis—Bleomycin-Induced Model

Idiopatic pulmonary fibrosis is a chronic lung disorder characterized by lung fibrosis. Bleomycin (BLM) has been widely used to induce lung fibrosis in rodents representing an experimental model of human IPF to study the potential efficacy of new therapies (Walters and Kleeberger 2008). To prepare the BLM-induced mouse model, C57BL/6J female mice at 10 weeks of age were subjected to intratracheal administration (introducing the drug directly in the trachea through inserting a needle in the mouth and throat) of 1.5 mg/kg (2.25-3U/kg) of BLM (Bleomycin Sulfate, Streptomyces verticillus, Merck Millipore), as described in the literature (Walters and Kleeberger 2008).

### 2.2—Pulmonary Fibrosis—ExoCell Functional Effect

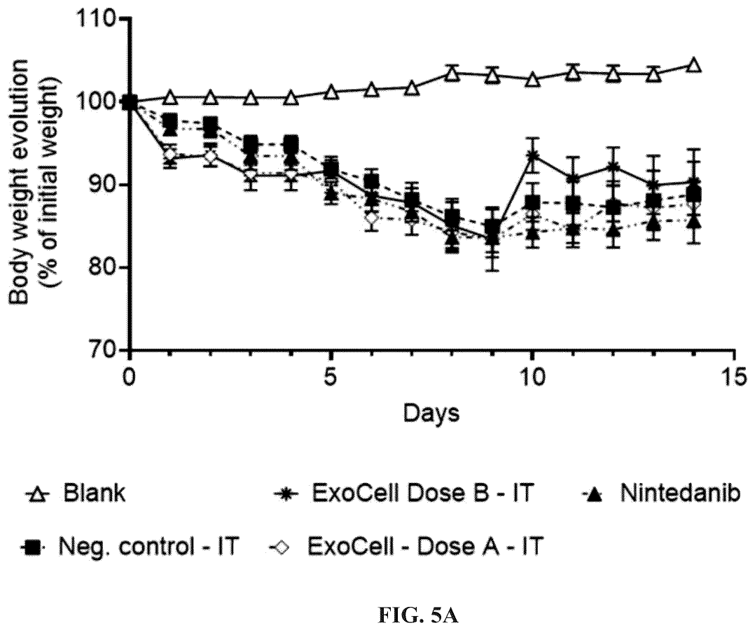

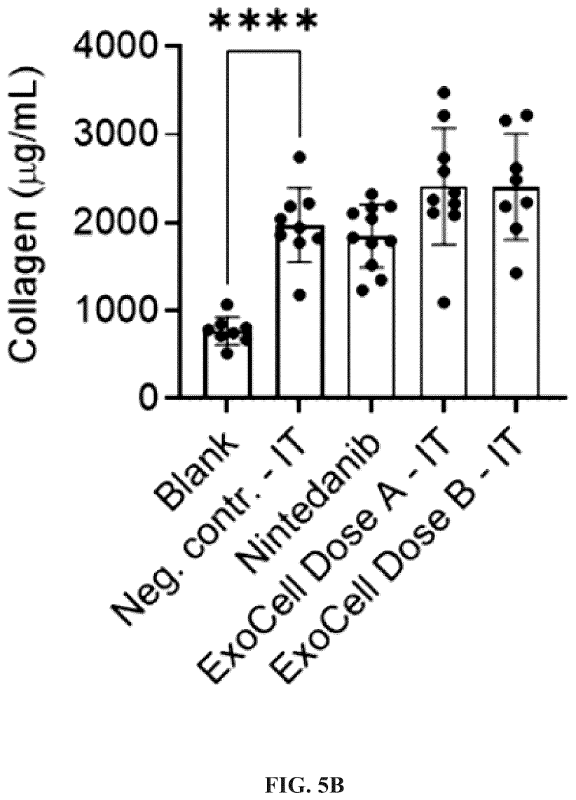

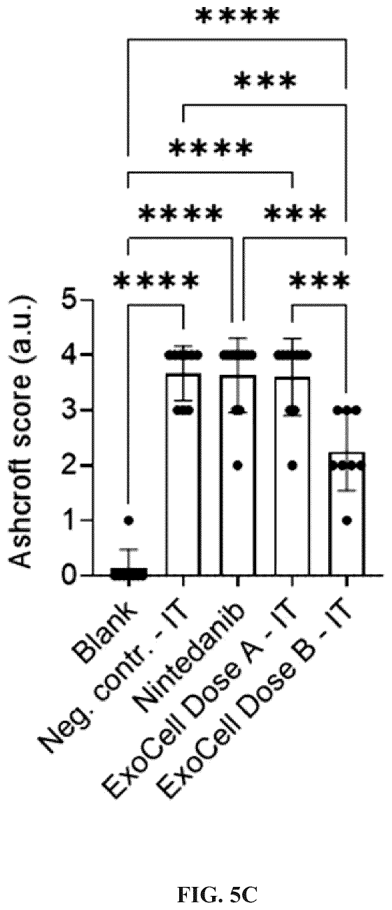

ExoCell was administered by intratracheal (IT) (Dose A—1×10⁹ particles and Dose B—1×10¹⁰ particles), starting on Day (D) 0, two hours before BLM administration until the end of the experiment on day 14 (12 animals). Mice were sacrificed at D14. Nintedanib, reference test compound (clinical comparator), was administered daily by oral route from D5 to D13. Healthy mice were used as blank (8 animals), and mice treated with BLM and PBS were used as negative control (12 animals). IPF symptoms include severe weight loss, thus mice weight was assessed and monitored daily. Mice receiving high dose (dose B) ExoCell IT tend to recover weight quicker than animals in the control groups (FIG. 5a) ExoCell showed a dose-dependent effect in this model.

Lung fibrosis in the alveolar septae and in the peri-bronchial area was assessed by histological analysis. At D14 mice were sacrificed by increasing concentration of CO₂ exposure. For lung collagen determination, after bronchoalveolar lavage (BAL) the whole lung was perfused with saline through the right heart ventricle to flush the vascular content and lungs were frozen at −80° C. Lung was homogenized in PBS by Ultra Turrax, centrifuged and the supernatant was discarded, and the pellet was resuspended in 1 mL PBS containing protease inhibitors (Roche). Following centrifugation, collagen content was determined by the Sircol assay (France Biochem Division, France). Collagen content increased in BLM treated mice comparing to healthy control animals (FIG. 5b).

After BAL and lung perfusion, the left lobe was directly immersed and fixed in 4% buffered formaldehyde (Fisher Scientific, ref. 15225582) for a minimum of 72 hours (h) and embedded in paraffin. 3 μm sections were stained with sirius red (SR) a marker of collagen fibres. For SR staining, slides were dehydrated in successive alcohol bath with increasing concentrations. Slides were immersed in Direct red solution for 30 min and rinsed in a bath of water, then immersed in Weigert's iron haematoxylin solution for 2 min. Lung sections were washed in water, hydrated in successive alcohol bath with decreasing concentrations and protected with coverslips. The collagen deposition located in the alveolar septae and in the peribronchial area was assessed by a semi-quantitative score—Ashcroft score—graded at 0 to 5 (Table I).

ExoCell dose B IT has a lower Ashcroft score at D14 comparing with negative control and Dose A (FIG. 5c). This data shows that exosome compositions of the present invention, when intratracheally administered (IT) accelerate recovery of mice with Bleomycin (BLM)-induced lung fibrosis in a dose-dependent fashion.

TABLE I
Semi-quantitative score for fibrosis - Ashcroft score

| Grade of fibrosis | Histological features |
|---|---|
| 0 | Normal lung |
| 1 | Minimal fibrosis thickening of alveolar |
| | or bronchiolar walls |
| 2 | Moderate thickening of walls without |
| | obvious damage |
| 3 | Moderate thickening of walls with lung |
| | structure damage |
| 4 | Increase fibrosis with lung structure damage |
| 5 | Severe distortion of structure and fibrotic area |
| | Total fibrous obliteration of the field |

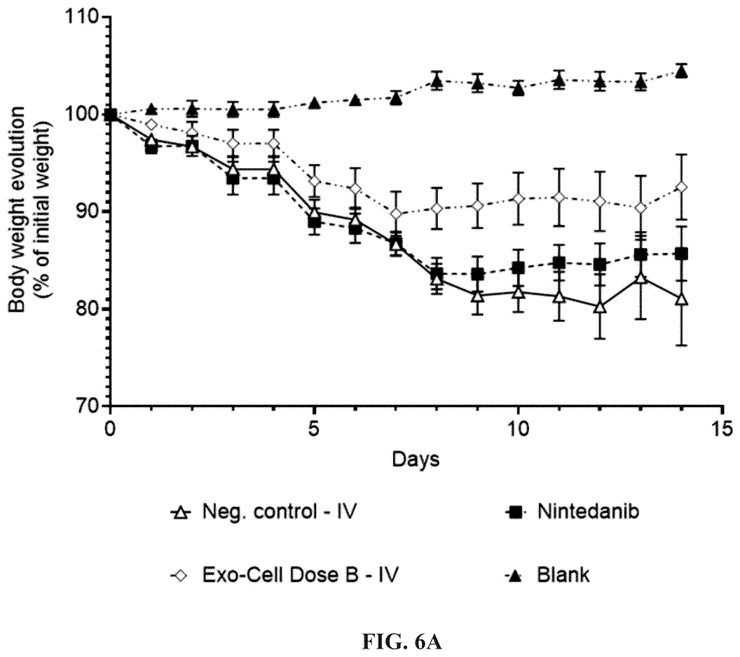

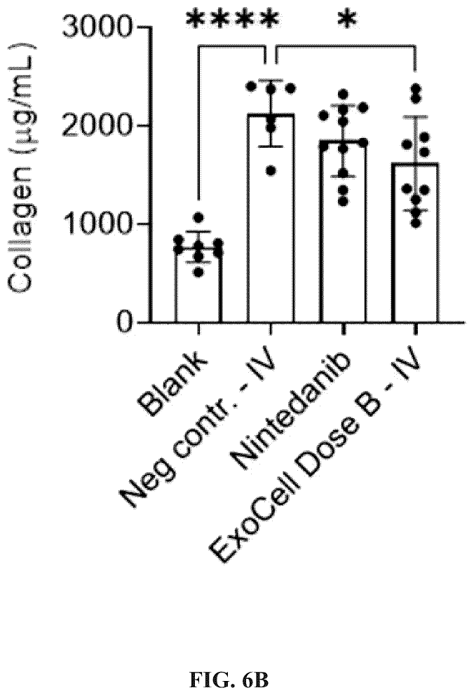

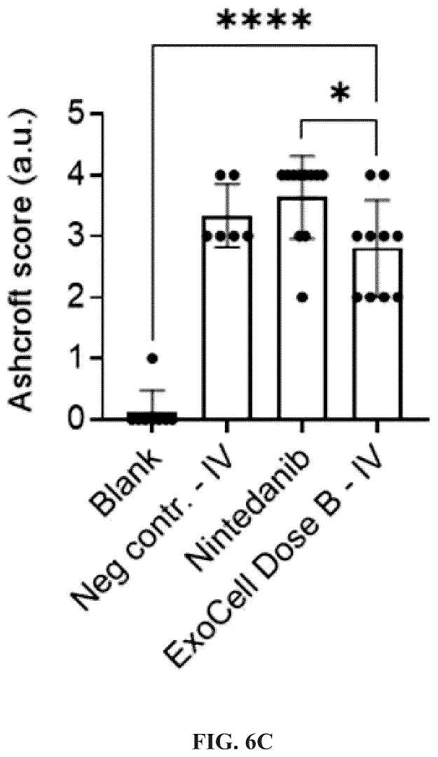

The composition of exosomes was administered intravenous (IV) (Dose A—1×10⁹ particles and Dose B—1×10¹⁰ particles), starting on Day (D) 0, two hours before BLM administration until the end of the experiment on day 14. Mice were monitored daily for body weight and clinical score. Mice receiving high dose (dose B) of ExoCell IV tend to recover weight quicker than animals in the control groups (FIG. 6a) ExoCell showed a dose-dependent effect in this model. Levels of lung collagen measured by Sircol assay (above described) were reduced in mice treated with Dose B (FIG. 6b) comparing to negative control (BLM-mice treated with PBS).

Moreover, the Ashcrof score tend to decrease in mice treated with exosome compositions (FIG. 6c).

In conclusion, this data shows that the intravenous (IV) administration of the exosome compositions of the invention prevents weight loss and collagen accumulation in mice with Bleomycin (BLM)-induced lung fibrosis.

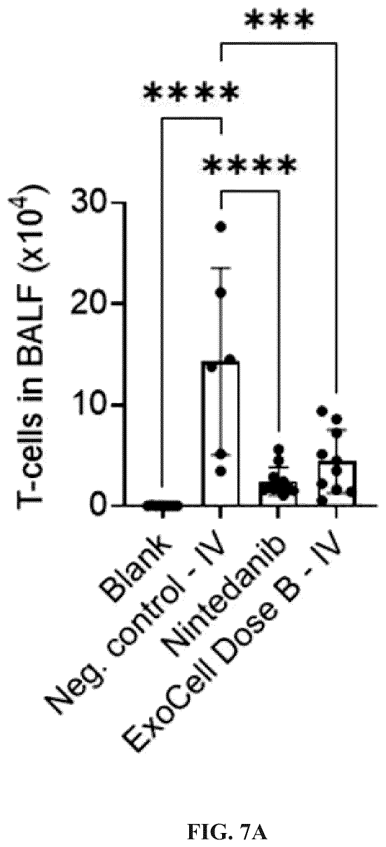

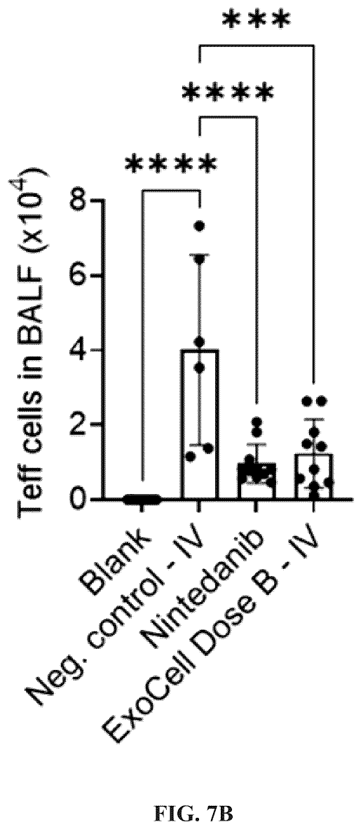

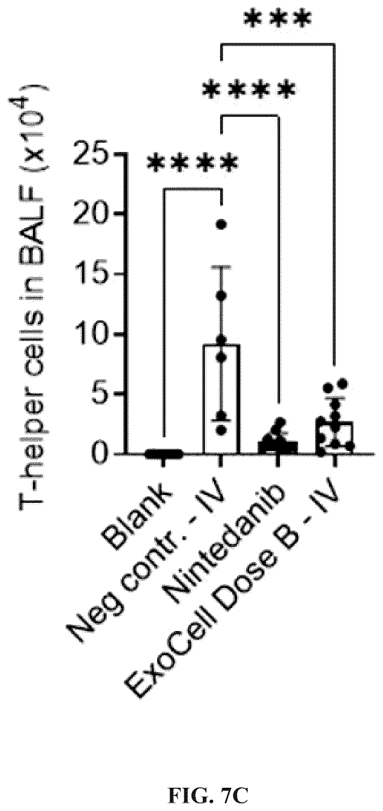

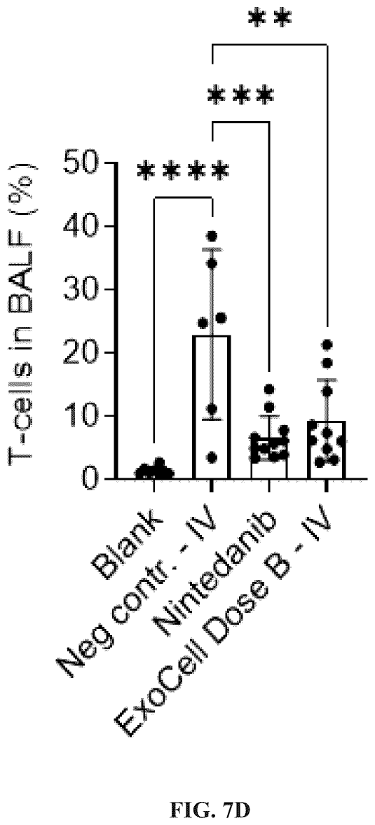

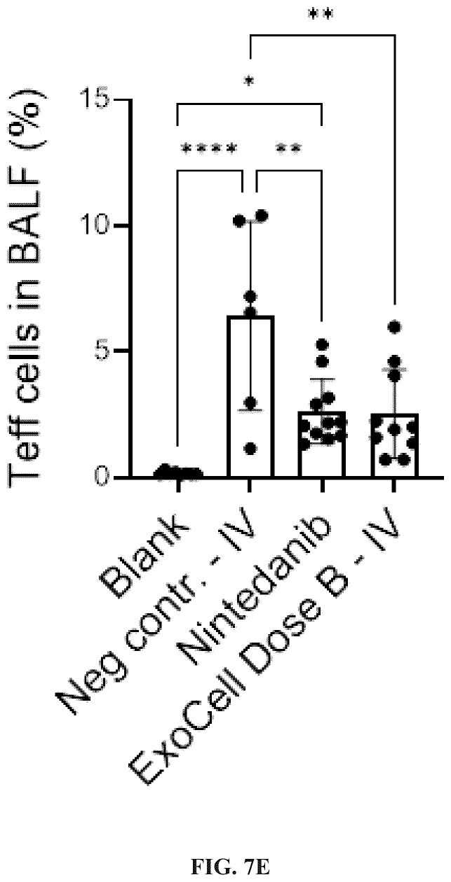

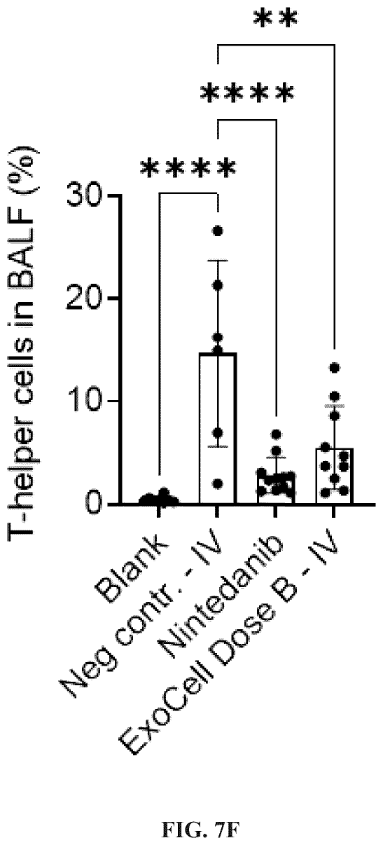

Effect of ExoCell compound on immune cell composition of bronchoalveolar lavage fluid (BALF) at D14. Lung fibrosis was induced in C57BL/6J mice following administration of BLM. Exosome composition at the doses of 1×10¹⁰ particles was administered by IV route every two days from DO to D12 after BLM administration. Cells isolated from BALF and digested lung (2×10⁶ cells/well) were suspended in fluorescence-activated cell sorting (FACS) buffer [PBS containing 3% Foetal Calf Serum (FCS), 2 mM EDTA] and labelled with live dead cell dye and specific surface antibodies (CD4 and CD8) and Fluorescence minus one (FMO) control. Flow cytometry analysis was performed on a BD Fortessa X-20 flow cytometer and the data were analysed with FlowJo software (Tree Star). Animals treated with ExoCell IV had significantly lower numbers (FIGS. 7 a,b and c) and percentages of total (FIG. 7 d,e and f) of CD4+ and CD8+ T-cells in bronchoalveolar lavage fluid (BALF), indicating that ExoCell's MoA in this model likely targets T-cells. CD4+ and CD8+ T-cells are associated with pulmonary fibrosis, CD4+ produce cytokines that can act profibrotically and the accumulation of CD8+ cells is related with pulmonary impairment (Luzina et al. 2008).

### 2.3—Acute Pulmonary Fibrosis—Lipopolysaccharides (LPS) Induced Mouse Model

To simulate human acute respiratory distress syndrome (ARDS), animal models should reproduce the acute injury to the epithelial and endothelial barriers in the lungs and the acute inflammatory response in the air spaces. One of the most used models is Lipopolysaccharide-induced Acute Lung Injury (LPS-induced ALI). The damage caused by LPS is characterized by an acute phase, including leukocyte influx, high levels of pro-inflammatory cytokines in BALF. Following the exposure of mice to an airway challenge of LPS (intranasally) a marked leukocyte infiltration (mainly neutrophils, but also lymphocytes and macrophages) in the BALF is induced, reaching a peak at 24 h post-stimulus. There are also increases in the pro-inflammatory cytokines TNFα, IL-6, IL-10 and IL-17A.

### Example 3. Mouse Model Preparation

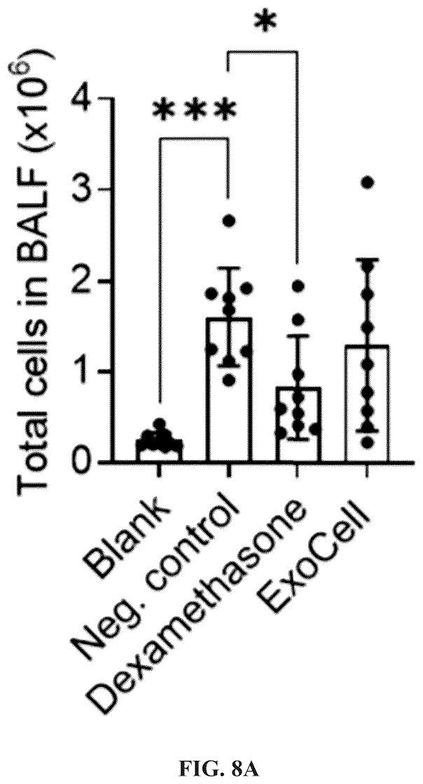

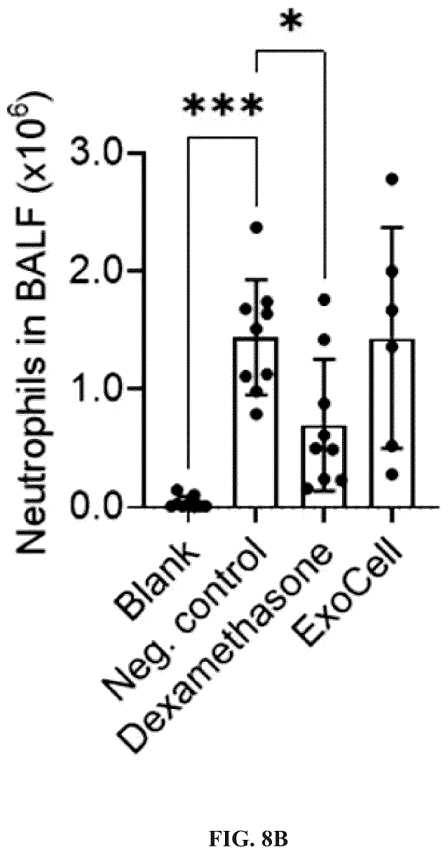

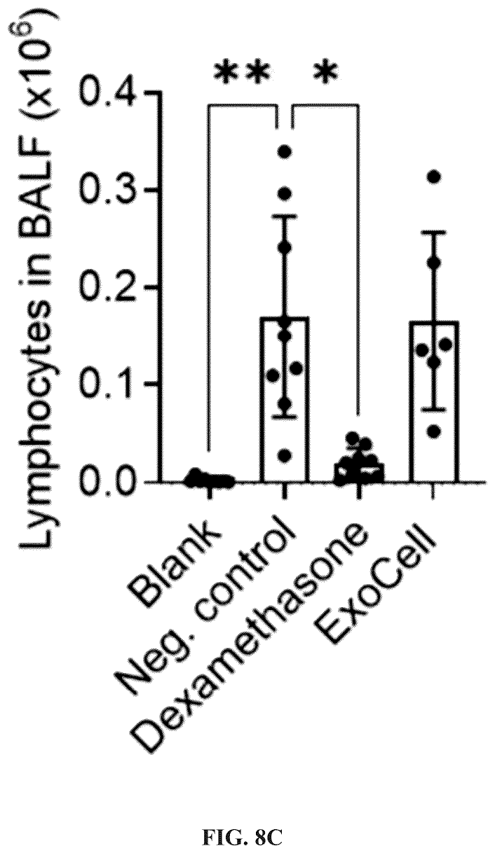

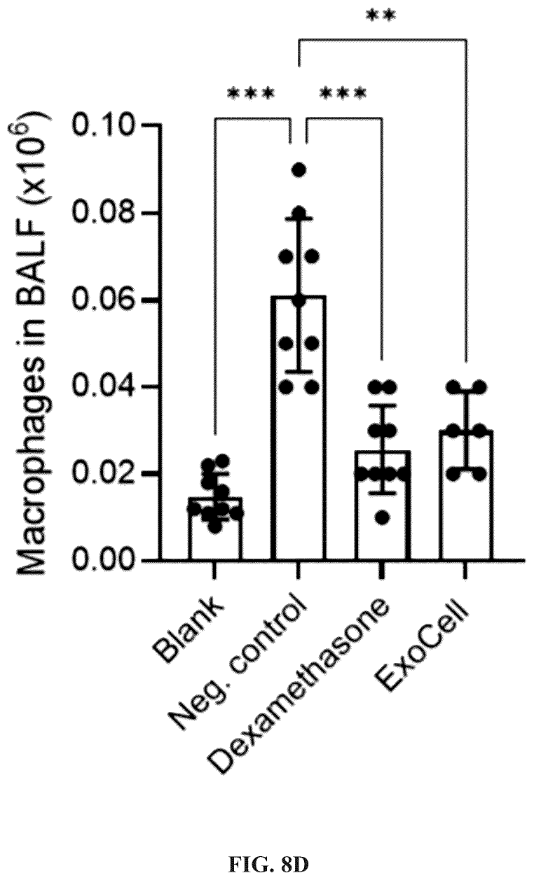

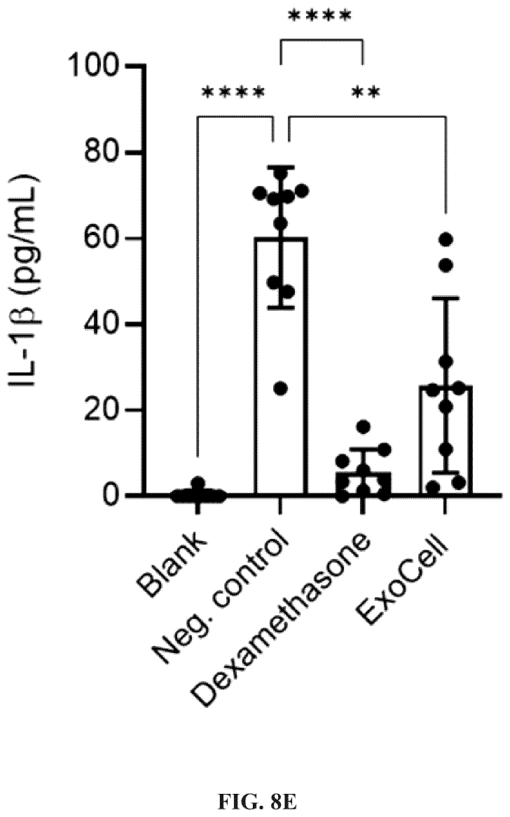

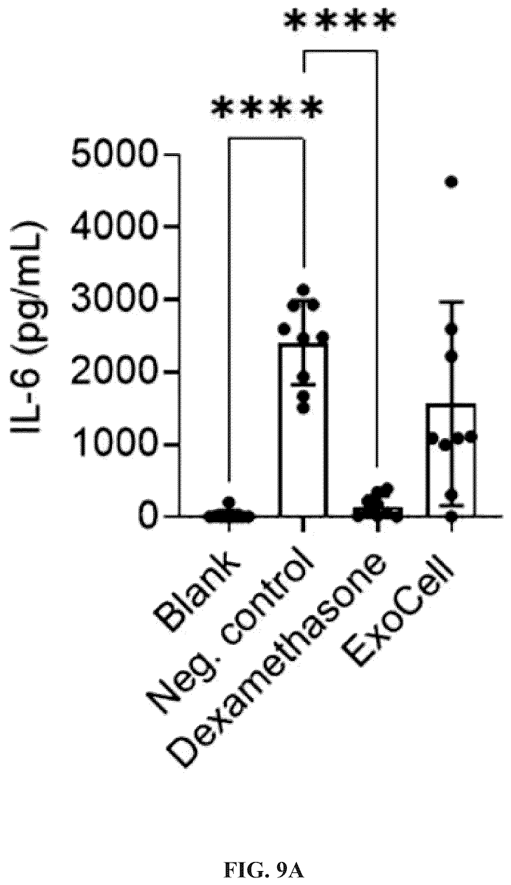

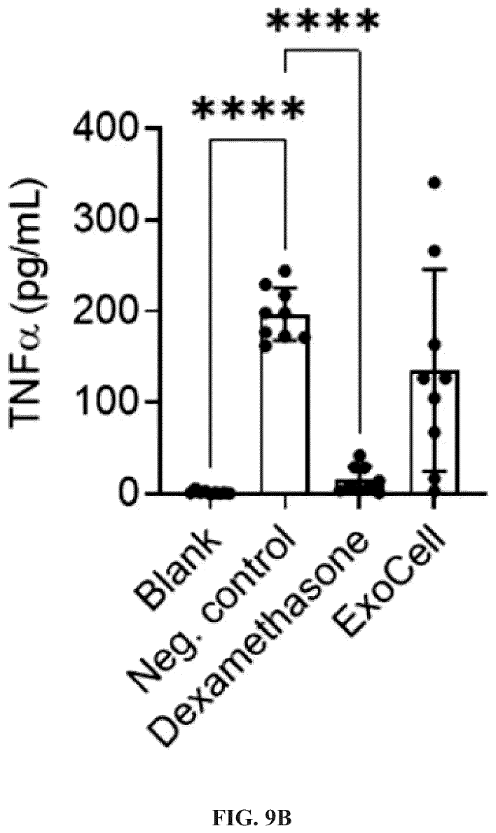

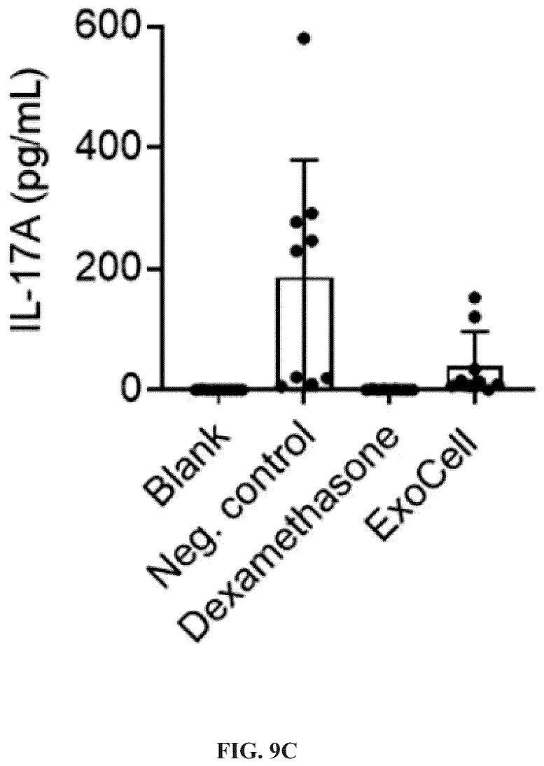

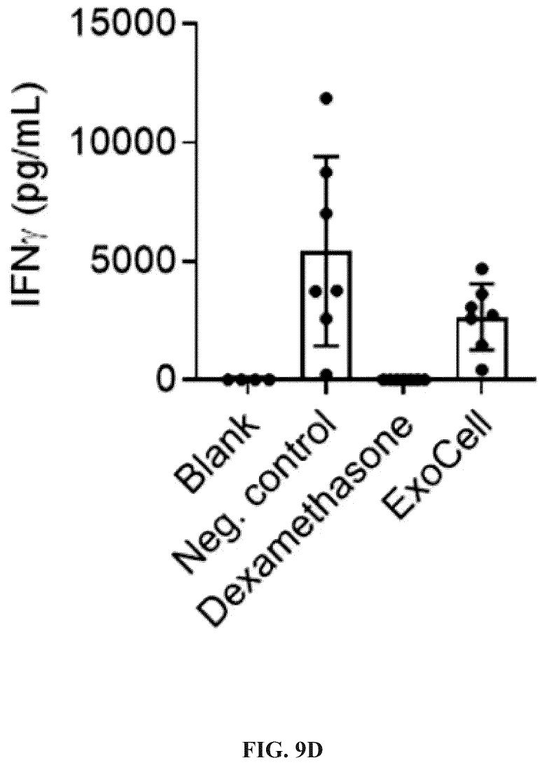

Lipopolysaccharides (LPS) was prepared immediately before treatment for intranasal challenge: 1 mg of LPS was dissolved in 4 mL of PBS to obtain a solution which concentration was 0.25 mg/mL. The animals were intranasally administered with 50 μl of this LPS solution and, therefore, mice that were challenged with LPS received a dose of 0.5 mg/kg. Briefly, mice are placed in a supine position, and given 50 microliter in drops of this LPS solution (0.25 mg/mL) and, therefore, mice that were challenged with LPS received a dose of 0.5 mg/kg through a micropipette. Animals were treated with 5 mg/kg of Dexamethasone 1 hour before LPS challenge as a reference compound (positive control). On day 0, animals were deeply anesthetized with isoflurane and intranasally administered with 50 μL of PBS (non-LPS challenged group 1) or LPS at 0.5 mg/kg dissolved in PBS. After intranasal instillation, mice were kept vertical for 1 min to ensure the correct distribution of LPS to the lungs. After LPS administration, every mouse was observed to monitor their normal breathing behaviour. A total of 1×10⁹ particles of ExoCell were administered once intranasally per animal one hour before LPS challenge. The administered intranasal volume was 50 μL. Briefly, mice are placed in a supine position, and given 50 microliter in drops of ExoCell through a micropipette. BALF samples were obtained after infusing lungs slowly with a 1 mL syringe (Injekt®-F Luer Solo, Braun) 0.3 mL PBS (with 2% foetal bovine serum, FBS) three times and recovering it by gentle aspiration after 30 seconds. Then BALF samples were centrifuged at 300 g for 8 min at 4° C. and the supernatants were transferred into new Eppendorf tubes and stored at −80° C. for cytokine analysis and cell pellets were processed for Flow Cytometry analysis. Flow Cytometry analysis was performed in leukocytes from BALF samples collected on day 1. Absolute cell counts were determined at the beginning of the processing and then cells were resuspended on either 50 or 100 μL of Stain Buffer depending on the cell concentration of each sample. Cell suspensions were stained with the appropriate antibodies' combination to assess frequencies of macrophages (CD45, CD11, F4/80) and neutrophils (CD45, CD11b and Ly6C). Then, samples were fixed in PBS with 1% formaldehyde and kept at 4° C. in darkness until the following day that they were acquired in a FACS Aria fusion cytometer (BD Biosciences). Raw data (FCS files) generated on the FACS Aria Fusion flow cytometer (FACS Diva software) was analysed using FCS Express software v 7.0 (DeNovo Software). Macrophages are specifically targeted by ExoCell in this model, the number of macrophages in BALF reduced in ExoCell treatment comparing to negative control (FIG. 8 d). Neutrophil and lymphocyte infiltration remains unaffected (FIGS. 8b and c). Cytokine multiplex analysis of IL-1β, TL-6, IL-10, IL-17A and TNF-α was carried out in supernatant samples collected 24 hours after LPS administration by using a Luminex kit (Panel MCYTOMAG-70k-05 Mouse MAG) in BALF. Data was acquired and processed with Luminex xPONENT software and analysed by MILLIPLEX Analyst 5.1 program. The threshold for an appropriate acquisition was established (>35 events). Samples following this criterium were included in the analysis. When results were below the limit of detection (<3.2 pg/ml), the concentration was considered as 0. The levels of IL-6 (FIG. 9 a), TNFα (FIG. 9 b), IL-17A (FIG. 9 c) and IFNγ (FIG. 9 d) in BALF reduced in ExoCell-treated animals, comparing with the non-treated (negative control) group. Additionally, IL-1β (proinflammatory cytokine) secretion is significantly reduced in BALF (FIG. 8 e).

1. A pharmaceutical composition comprising exosomes (ExoCell) secreted by umbilical cord blood mononuclear cells (UCBMNCs) exposed to hypoxia conditions before the separation of the exosomes from the UCBMNCs, said composition is applicable in the prevention or treatment of fibrotic disorders selected from the group of: skin fibrosis, pulmonary fibrosis, nephrogenic fibrosis, and liver fibrosis, wherein:
skin fibrosis are scleroderma, keloids and scars,
pulmonary fibrosis are chronic obstructive pulmonary disease (COPD), respiratory distress syndrome (ARDS), sarcoidosis, idiopathic pulmonary fibrosis (IPF), scleroderma,
liver fibrosis are sarcoidosis, liver cirrhosis,
fibrotic lesions are systemic sclerosis, nephrogenic systemic fibrosis and liver fibrosis,
the fibrotic disorders are of inflammatory, autoimmune, infections, injury, or idiopathic origin, and
wherein said compositions are prepared to be applied via intratracheal (IT), intranasal or micro-sprayer with intranasal (MS) or intravenous (IV).

2. A method for preventing or treating a fibrotic disorder using the composition of claim 1, wherein fibrotic disorders are of inflammatory origin.

3. A method for preventing or treating a fibrotic disorder using the composition of claim 1, wherein fibrotic disorders are of autoimmune origin.

4. A method for preventing or treating a fibrotic disorder using the composition of claim 1, wherein fibrotic disorders are lung viral or bacterial infections.

5. A method for preventing or treating a fibrotic disorder using the composition of claim 1, wherein fibrotic disorders are of injury origin.

6. A method for preventing or treating a fibrotic disorder using the composition of claim 1, wherein the said composition is in the form of powder, microparticles, aerosol, liquid suspension, or liquid solution.

7. A method for preventing or treating a fibrotic disorder using the composition of claim 1, comprised in a device, wherein the device is one of a metered dose inhaler, a dry powder inhaler, a container for spray for nasal administration or nebulization.
